(19) United States
(12) Patent Application Publication
(10) Pub. No.: US 2017/0105946 A1
(43) Pub. Date: Apr. 20, 2017

(54) HERBAL COMPOSITIONS INCLUDING CANNABIDIOL TO ENHANCE THE SEXUAL EXPERIENCE

(71) Applicant: Matthew J. Stepovich, Santa Cruz, CA (US)

(72) Inventor: Matthew J. Stepovich, Santa Cruz, CA (US)

(21) Appl. No.: 14/886,101

(22) Filed: Oct. 19, 2015

(51) Int. Cl. A61K 31/05

(52) U.S. Cl. CPC A61K 31/05

(57) ABSTRACT

The present invention provides herbal compositions comprising cannabidiol (CBD) for enhancing the sexual experience. In accordance with the invention, a physiologically acceptable composition including an emulsion of cannabidiol and a carrier substance is delivered to the skin, one or membranes or other surfaces of the human body where absorption of the herbal composition can occur. In some embodiments of the invention the delivery mechanism includes an apparatus, including but not limited to dissolving strips, vaporizers, pump sprays, single-use foil tubes, and condoms. The delivery occurs prior to or during sexual activity. Some embodiments of the invention provide carrier substances that will improve the effectiveness of the CBD in enhancing the sexual experience, and that may improve the experience in other ways, such as by adding flavors or fragrances.

### CROSS-REFERENCE TO RELATED APPLICATIONS

This application claims priority from and incorporates herein by reference in its entirety U.S. Non-Provisional Patent Application No. 62/122,409 dated Oct. 18, 2014.

### BACKGROUND OF THE INVENTION

Field of the Invention

The disclosed embodiments relate generally to the field of enhancement of sexual pleasure of human couples.

Description of Related Art

It is generally known that the pleasure derived from sexual encounters may vary from person to person and from encounter to encounter. The variables that may affect the experience are numerous, from physiological and psychological, to environmental.

In the physiological and psychological realm, herbal compositions may make it more likely that a person will have a gratifying sexual experience and may strengthen the climax or orgasm.

In the environmental realm, the timing of the climax may affect the sense of pleasure and the strength of the orgasm. It is known that delaying the climax tends to make the climax stronger, and it is also accepted that by timing the climax to be simultaneous with that of a partner also enhances the sexual experience.

Hence, a first factor in enhancing the sexual experience may be to influence the strength of the orgasm physiologically, while a second factor is to enhance the parties' ability to delay and otherwise optimize the timing of his or her climax.

There have been various efforts to alter the timing of climax using many substances, mostly focused on delaying the male climax by reducing the sensations received by the male during the sexual encounter.

### Anesthetic Creams

Many of these efforts have employed local anesthetics such as lidocaine or prilocaine in an emulsion applied as a cream. Generally, creams have been effective in delaying climax and do not have significant adverse side effects, but the numbing affect of these creams is thought to reduce the sexual pleasure for one or both participants making topical anesthetics a less attractive alternative than a substance that had little or no effect on the pleasure or sensation experienced by either participant.

### Antidepressants

A side effect of certain prescription antidepressants is a delay in orgasm. These drugs therefore may improve the timing of climax, however none has been approved for this indication by the FDA and they are intended and expected primarily to have an effect on personality.

### Cannabidiol

A wide variety of non-prescription CBD-based products are available today for uses ranging from cosmetics, to joint comfort, and improvement in post-sports recovery time.

In the authors' investigations, the application of an herbal CBD emulsion, as described in the below disclosure, prior to a sexual experience has been reported to enhance the strength of orgasms, to delay and improve the ability of the male to control the timing of climax, and may also improve the female ability to control climax. Unlike the use of analgesics, the improvements from CBD application are reported to have been achieved without the negative side effect of numbness of the parts of the body to which the CBD emulsion has been applied.

The herbal CBD emulsion that is the subject of the below disclosure has been reported to significantly enhance the sexual experience for both partners.

### SUMMARY OF THE INVENTION

Cannabinoids are a class of compounds or herbs present in Indian cannabis (cannabis sativa), including about sixty different molecules. One of these is Cannabidiol (CBD). CBD is generally derived from the hemp varieties of the cannabis sativa plant, as distinguished from the marijuana variety of the cannabis sativa plant. The marijuana variety of the cannabis sativa plant has a high concentration of tetrahydrocannabinol (THC). The hemp varieties have high concentrations of CBD, but almost no THC.

The inventors have found that herbal compositions of the CBDs found in the hemp variety of the cannabis sativa plant are useful in enhancing the sexual experience of men and women.

The invention disclosed herein is an herbal composition containing Cannabidiol (CBD) used to enhance the sexual experience. The herbal composition of CBD can be in the form of extract or isolate from the hemp variety of the cannabis sativa plant. The herbal composition may include CBD and an emulsifying substance that facilitates the application of the herbal CBD to the skin, mucous membranes, or another part of the body and the CBD's absorption.

### BRIEF DESCRIPTION OF THE DRAWINGS

FIG. 1 is a diagrammatic representation of a vaporizing e-cigarette type CBD vapor delivery apparatus.

FIG. 2 is a diagrammatic representation of a dissolving strip CBD emulsion type delivery apparatus.

FIG. 3 is a diagrammatic representation of a condom, partially cut away, showing a coating of a CBD emulsion on the outside.

### DETAILED DESCRIPTION

The invention described herein involves the application of an herbal emulsion including Cannabidiol (CBD), a well-known non-scheduled chemical substance, to the skin or other surface of the human body of a sexual partner, often but not exclusively to the penis or the vagina. The purpose is to enhance the pleasure of the experience by (a) giving one or both partners a greater degree of control over timing of his or her climax, (b) by delaying the timing of the male participant's climax, by increasing the intensity of the climax, or (c) some or all of the above.

The herbal emulsion may include CBD suspended or mixed in an ointment, gel, cream, vapor, or other carrier (together, herein, the “CBD emulsion”), which CBD emulsion makes it more conveniently applied to the skin, membrane or other absorptive body structure and may improve its effectiveness.

Human skin and membranes include layers of important lipid barriers, which function to protect the body from the entry of harmful foreign substances encountered in the environment. In one embodiment of the invention, the CBD emulsion may include a carrier that is chosen for its lipid solubility, which increases the amount of CBD that crosses the skin's lipid barrier, improving the ability of the CBD emulsion to enter the body's tissues and enhance the sexual experience.

In another embodiment of the invention, the CBD emulsion includes dimethyl sulfoxide (DMSO), which also enhances absorption.

In additional embodiments of the invention, the carriers are made up in whole or in part of plant based oils that are known to have antimicrobial, antiviral, and/or antifungal properties. In such embodiments, these oils would include but would not be limited to tea tree oil, oreganum (oregano) oil, lavender oil, geranium oil, lemon oil, eugenol (clove oil), salvia officinalis (sage) oil, cinnamon oil, lemongrass oil, thyme oil, mint oil, geranium oil, cinnamon oil, rosemary oil, coconut oil^{1,2}, or the oil of cannabis sativa seeds. Embodiments may contain mixtures of these and other oils, as well as substances that are preservatives, and/or that provide fragrance, color, or flavor. ¹Ogbolu D O, Om A A, Daini O A, Olokolo A P, In vitro antimicrobial properties of coconut oil on Candida species in Ibada, Nigeria, J Med Food, Vol. 10, No. 2, PP. 384-87 (2007).²Nevin K G, Rajamohan T, Effect of topical application of virgin coconut oil on skin components and antioxidant status during dermal wound healing in young rats. Skin Pharmacol Physiol, Vol. 23, No. 6, PP. 290-97 (2010).

With respect to coconut oil, it may be beneficial to use coconut oil that is pressed mechanically from copra with sufficient force to produce oil that is liquid at body temperature, or approximately 37° C. (98.6° F.). Similarly, other carrier oils or emulsions, including such oils should ideally be liquid at body temperature to enhance CBD transfer through the lipid barriers of the skin.

In some embodiments of the invention, an emulsion that is in solid or gel form at room temperature (approximately 21° C. or 70° F.) but becomes liquid at a convenient temperature that is between room temperature and body temperature. In this embodiment, the CBD emulsion is more easily applied in the gel or cream state, but as it quickly warms to body temperature the CBD becomes more a liquid making it more effective at crossing the lipid barrier because the CBD emulsion is in liquid state.

In additional embodiments of the invention, natural oils that DO NOT have antimicrobial or antifungal properties may also be used as carries for CBD.

In an additional embodiment of the invention, the CBD emulsion consists of 1% or more of an emulsifying oil that is antimicrobial and/or antifungal, plus one or more other carriers and CBD. In this embodiment, the addition of the antimicrobial/antifungal oil reduces or eliminates the need for synthetic preservatives in the CBD emulsion.

In other embodiments of the invention, synthetic or other preservatives that are safe for human use may be combined with the CBD emulsion.

In some embodiments of the invention, the CBD emulsion is a cream, gel or liquid that is applied by the sexual participants to their own skin, or their sexual partner's skin.

In some embodiments of the invention, the CBD emulsion is on the inside of a prophylactic condom, such that the process of placing the condom on the penis also brings the CBD emulsion into contact with the skin of the penis.

In some embodiments of the invention, the CBD emulsion is contained in a “break pack” along with the condom in one single package, such that the CBD emulsion would not be in contact with the condom material during manufacturing, storage, or transport, but would only come in contact with the condom material just prior to the condom being used. This may enhance the manufacturability of a sterile condom and a sterile CBD emulsion product, and it would prevent the long or medium term effects that the condom or the CBD emulsion materials may have on each other.

In some embodiments of the invention, the condom would be made of latex rubber. In other embodiments the condom materials would be non-latex.

In some embodiments of the invention, the CBD emulsion would be both on the inside and the outside surfaces of the condom, so that both the male and female receive the enhancement that the CBD emulsion can provide to their experience.

In some embodiments of the invention, the CBD emulsion may be in a form designed to be applied using a pump spray device, which may be an element of the invention.

In other embodiments the CBD Emulsion may be designed to be applied using an aerosol spray device, which device may also be an element of the invention.

In other embodiments the CBD Emulsion may be designed to be inhaled using a vaporizer device, some of which are referred to as e-cigarettes, which devices may also be an element of the invention. In one embodiment there is a vaporizer having a mouth piece, a chamber for holding a quantity of the CBD emulsion, and a battery powered heating element. The CBD emulsion is drawn from the chamber and heated by the heating element to a vaporization temperature at which point the vapors of the CBD emulsion are inhaled in by a user.

In other embodiments the CBD Emulsion may be designed to be delivered orally using a dissolving strip device, or a mucoadhesive film device, which device may also be an element of the invention.

In other embodiments the CBD Emulsion may be designed to be delivered using a transdermal patch device, which device may also be an element of the invention.

In some embodiments of the invention, the CBC emulsion would contain a penetration enhancer, including but not limited to DMSO, or substances that assist the CBD in crossing the body's lipid barriers.

In some embodiments of the invention, the CBD emulsion would include a fragrance, including but not limited to lavender, cinnamon, peppermint, and vanilla.

In some embodiments of the invention, the CBD emulsion would include a flavoring.

Several cannabinoid formulations with varying concentrations of CBD are contemplated, from 0.25% CBD to 30% CBD.

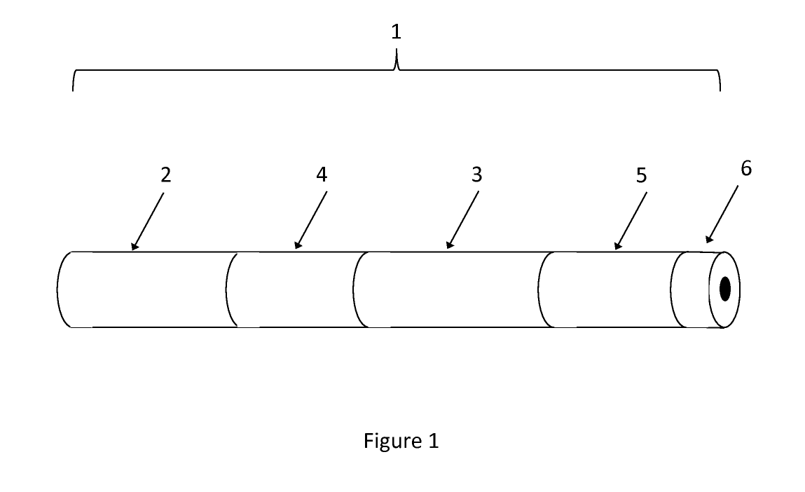

Referring to FIG. 1, one embodiment of the invention uses an e-cigarette vaporizer to deliver vaporized CBD when the user inhales. The e-cigarette vaporizer 1 includes a battery 2 that powers a vaporizer 3. A reservoir 4 holds the CBD formulation to be vaporized by the vaporizer 3. When the user puts negative pressure on mouthpiece 6, CBD is drawn from reservoir 4 into vaporizer 3, which is activated to heat the CBD, which becomes a vapor and enters vapor chamber 5. As the user continues to draw on mouthpiece 6, vaporized CBD is inhaled by the user through mouthpiece 6. Reservoir 4 may be refillable, or it may include a replaceable CBD cartridge (not shown).

The CBD solution in one embodiment of the invention described in FIG. 1 includes 0-50% CBD and 0-75% propylene glycol. Other embodiments include 0-30% CBD, 0-20% ethanol, 0-20% aqueous glycerin, 0-65% propylene glycol, 0-25% vegetable glycerin, 0-5% flavoring. Other embodiments may include the above with the addition of 0-2% tetrahydrocannabinol (THC).

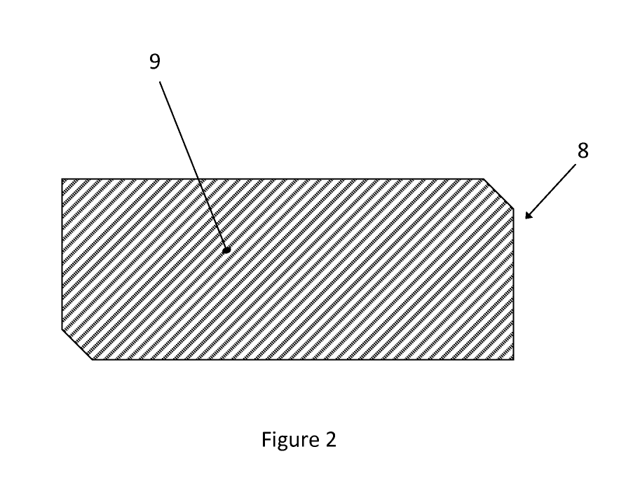

Referring to FIG. 2, a device 7 contains a wafer-shaped dissolving food grade carrier matrix 8 that breaks down when it is placed in contact with saliva (not shown), releasing CBD extract 9 with which dissolving food grade carrier matrix 8 has been impregnated. The device is placed in the mouth sublingually or buccally, in contact with the oral mucosa (not shown). As dissolving food grade carrier matrix 8 dissolves due to the contact with saliva, it released CBD, which permeate or be absorbed through the mucosa and into the body. In this embodiment, the CBD extract amounts to 0-50% by weight, with 0-99% dissolving food grade carrier. Other embodiments may include the above, plus optionally, additives from the following groups: fillers, colors, flavors, fragrances, emulsifiers, plasticizers, sweeteners, preservatives, absorption and permeation enhancing substances, pH regulators and antioxidants.

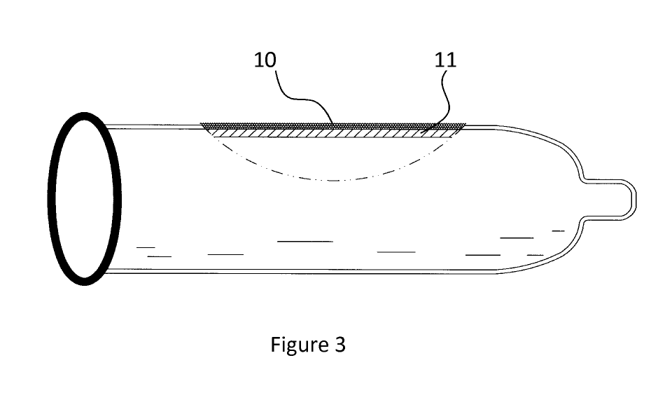

Referring to FIG. 3, a condom is represented having a outer layer 10 of latex or other flexible material typically used in prophylactic condoms and also having an inner layer 11 in some or all of the condom's inner surface comprising a CBD solution.

In some or all of the inventions described above, some amount of THC may be present in the CBD emulsion, or the emulsion may be THC-free.

Although the invention has been described with reference to specific exemplary embodiments of the invention thereof, it will be evident that various modifications and changes may be made thereto without departing from the broader spirit and scope of the invention. The specification and drawings are, accordingly, to be regarded in an illustrative rather than a restrictive sense.

1. The use of cannabidiol for the manufacture of a supplement to enhance the sexual experience.

2. A substance containing cannabidiol delivered to a surface of the human body to enhance the sexual experience.

3. A method of enhancing the sexual experience comprising the step of delivering an emulsion of CBD and a carrier substance to a surface the human body.

4. The method of claim 3 wherein the surface is a mucosal membrane.

5. The method of claim 3 wherein the surface is lung tissue.

6. The method of claim 3 wherein the emulsion is delivered via a patch;

7. The method of claim 3 wherein the emulsion is delivered via a spray device;

8. The method of claim 3 wherein the emulsion is delivered using a vaporizer;

9. The method of claim 3 wherein the emulsion is applied using dissolving oral strip.

10. The method of claim 3 wherein the emulsion is applied using a dissolving oral tablet.

11. The method of claim 3 wherein the emulsion is contained in a condom.

12. The method of claim 3 wherein the emulsion is in a separate package along with the condom, which separate package is opened prior to using the condom, releasing the emulsion so that it can be applied when the condom is placed on the user.

13. The method of claim 3 wherein the emulsion includes a synthetic or a plant-derived oil.

14. The method of claim 13, wherein the oil is an antimicrobial or antifungal.

15. The method of claim 3 wherein the emulsion includes a preservative.

16. A method of enhancing the sexual experience comprising a step administering a substance containing cannabidiol.

17. The method of claim 16 wherein the substance is inhaled.

18. The method of claim 16 wherein the substance is vaporized and then inhaled.

19. The method of claim 16 wherein the substance is placed on a strip that is applied to oral mucosa.

20. The method of claim 16 wherein the substance is in a dissolving oral tabled.

21. The method of claim 16 wherein the substance is aerosolized and inhaled or sprayed into the nasal cavity.

22. A method for enhancing the sexual experience, comprising administering to a subject an effective amount of a cannabidiol compound wherein the cannabidiol compound is part of a preparation being selected from the group consisting of a tablet, a capsule, a dissolving oral strip, a dissolving oral capsule, a vapor, a cream, a gel, and a suspension in a solution.

23. The method of claim 3 wherein the carrier substance contains a penetration enhancer.

24. A method for enhancing the sexual experience comprising administering to a subject a composition comprising cannabidiol, absent THC and THCV.
